(19) United States
(12) Patent Application Publication
(10) Pub. No.: US 2013/0345306 A1
(43) Pub. Date: Dec. 26, 2013

(54) IDENTIFICATION OF C3 COMPLEMENT MOLECULE AT THE SITE OF INJURY TO BLOOD VESSELS IN THE RETINA OF OXYGEN EXPOSED ANIMALS

(71) Applicants: John T. FLYNN, Elmsford, NY (US); Quan WEN, New York, NY (US); Takayuki NAGASAKI, New York, NY (US)

(72) Inventors: John T. FLYNN, Elmsford, NY (US); Quan WEN, New York, NY (US); Takayuki NAGASAKI, New York, NY (US)

(73) Assignee: THE TRUSTEES OF COLUMBIA UNIVERSITY IN THE CITY OF NEW YORK, New York, NY (US)

(21) Appl. No.: 14/012,833

(22) Filed: Aug. 28, 2013

Related U.S. Application Data
(63) Continuation-in-part of application No. PCT/US2012/026977, filed on Feb. 28, 2012.
(60) Provisional application No. 61/447,253, filed on Feb. 28, 2011.

(51) Int. Cl. A61K 31/197; G01N 33/50

(52) U.S. Cl. CPC A61K 31/197; G01N 33/5088
USPC 514/563; 435/29

(57) ABSTRACT

The invention provides methods to treat oxygen-induced retinopathy using complement factor C3aR antagonists.

This application is a continuation-in-part of International Application No. PCT/US2012/026977 filed on Feb. 28, 2012, which claims the benefit of priority of U.S. Ser. No. 61/447,253 filed on Feb. 28, 2011, the contents of which are hereby incorporated in their entirety.

All patents, patent applications and publications cited herein are hereby incorporated by reference in their entirety. The contents of all of these publications in their entireties are hereby incorporated by reference into this application in order to more fully describe the state of the art as known to those skilled therein as of the date of the invention described and claimed herein.

### BACKGROUND

The Retinopathy of Prematurity (ROP) disease is a scourge of blindness during infancy. Now world-wide in its distribution and seen in its most virulent forms in the tiniest, very youngest, most fragile and innocent of our species, born as they are barely clinging to life at it roughest edge. Its therapy today although successful in the narrowest sense is largely destructive of normal retinal tissue blunting the VEGF signal by destroying neurons and glia by laser or destroying blood vessels employing an anti-cancer drug Avastin. Provided herein are methods for the development of a therapy for ROP. In certain embodiments, the therapeutic methods prevent ROP. In other embodiments, the methods contribute to treatment of ROP symptoms.

### SUMMARY

Described herein is the role that the active Complement C3 system plays in the OIR and particularly its effect in the apoptosis of the capillary bed. This is likely the fundamental set of events triggering the VEGF system to start the cycle of neovascularization leading to retinal detachment, visual loss and blindness. Described herein are methods of blocking the C3 receptor on the cell membrane with an agent, a non-limiting example of such agent is SB290157, and thereby leading to capillary bed preservation and preventing or reducing the cycle of ischemia, overwhelming VEGF signaling, new abnormal vessel growth, traction detachment and vision loss.

In certain aspects the invention demonstrates the role that SB290157(Ames et al. 2001), a small non peptide molecule Receptor Antagonist (RA) has in inhibiting the damaging effect of Complement C3a on the capillary vasculature exposed to 75% oxygen injury in newborn mice from DOL 7-12. The molecule SB290157 blocks the C3 receptor on the endothelial cell membrane thereby preventing the C3a active forms from destroying the endothelium of the capillary bed. In certain aspects the invention provides methods of treating, preventing or reducing oxygen induced capillary bed damage, for example but not limited to the eye, comprising administering to a subject complement antagonist, wherein in a non-limiting example, the complement antagonist is SB290157.

In certain aspects the invention provides methods of treating, preventing or reducing oxygen induced neovascularization, for example but not limited to the eye, comprising administering to a subject complement antagonist, wherein in a non-limiting example, the complement antagonist is SB290157.

In certain aspects, the invention provides a method to treat oxygen-induced retinopathy in a subject comprising administering to a subject in need thereof a therapeutic amount of an agent which inhibits complement activation. In certain aspects, the invention provides a method to prevent oxygen-induced retinopathy in a subject comprising administering to a subject in need thereof a therapeutic amount of an agent which inhibits complement activation.

In certain aspects, the invention provides a method to treat oxygen-induced capillary bed damage in the retina of a subject comprising administering to a subject in need thereof a therapeutic amount of an agent which inhibits complement activation. In certain aspects, the invention provides a method to prevent oxygen-induced capillary bed damage in the retina of a subject comprising administering to a subject in need thereof a therapeutic amount of an agent which inhibits complement activation.

In certain aspects, the invention provides a method to treat a disease or disorder of abnormal neovascularization due to oxygen induced toxicity in a subject comprising administering to a subject in need thereof a therapeutic amount of an agent which inhibits complement activation. In certain aspects, the invention provides a method to prevent abnormal neovascularization due to oxygen induced toxicity in a subject comprising administering to a subject in need thereof a therapeutic amount of an agent which inhibits complement activation. In certain embodiments, the abnormal neovascularization is in the eye.

In certain aspects, the invention provides a method to treat ROP comprising administering to a subject in need thereof a therapeutic amount of an agent which inhibits complement activation.

In certain aspects the invention provides methods of treating, preventing or reducing oxygen-induced retinopathy, oxygen-induced capillary bed damage, disease or disorder of abnormal neovascularization due to oxygen induced toxicity, or ROP, or any combination thereof, the method comprising administering to a subject in need thereof a therapeutic amount of SB290157.

In a non-limiting embodiment, the complement component which is targeted for inhibition is C3a receptor. In certain aspects, the invention provides therapeutic methods as disclosed herein, wherein the agent is SB290157.

In certain embodiments of the methods, the subject is a human. In certain embodiments, the subject is in need of treatment. In certain embodiments the subject is diagnosed or suspected of having an ocular disorder of vascularization. In certain embodiments, the subject is diagnosed or is suspected of having ROP. In certain embodiments the subject is an infant who is born prematurely. In certain embodiments the infant is born at about 24, about 25, about 26, about 27, about 28, about 29, about 30, about 31, about 32 about 33, about 34, about 36, or about 37 weeks of gestation. In certain embodiments the infant is born at about 24-33, 24-32, 24-31, 24-30, 24-29, 24-28, 24-27, 24-26, or 24-25 weeks of gestation. In certain embodiments the infant is born at about 25-33, 25-32, 25-31, 25-30, 25-29, 25-28, 25-27, or 25-26, weeks of gestation. In certain embodiments the infant is born at about 26-33, 26-32, 26-31, 26-30, 26-29, 26-28, or 26-27, weeks of gestation. In certain embodiments the infant is born at about 27-33, 27-32, 27-31, 27-30, 27-29, or 27-28, weeks of gestation. In certain embodiments the infant is born at about 28-33, 28-32, 28-31, 28-30, or 28-29, weeks of gestation. In certain embodiments the infant is born at about 29-33, 29-32, 29-31, or 29-30, weeks of gestation. In certain embodiments the infant is born at about 30-33, 30-32, or 30-31, weeks of gestation. In certain embodiments the infant is born at about 31-33 or 31-32, weeks of gestation.

This discovery opens the door to the role played by the immune system (C3 Complement) to the damage and repair of oxygen toxicity to developing vascular tissues. Knowledge of this immunological response to the injury was not known prior to this experiment. There are agents, including but not limited to pharmacological agents, which can modify and control the immune response much as occurs in transplant and rejection immunology. This line of investigation might lead directly to a pharmacological treatment of Oxygen-induced retinopathy (OIR) in the mouse and also Retinopathy of Prematurity (ROP) in the premature infant. The OIR model does specify loci of injury (PUFA) in the cell wall and time course of the appearance of the immune “first responder” and the role it plays throughout the injury and repair.

Described herein are non-limiting embodiments of method which provide the opportunity to begin to test known and as yet unknown pharmacological agents to begin to ameliorate and modify as far as possible the damage done by the toxic agent (Oxygen) and the immune agent(s) coping with it. This is not just limited to OIR but it is conceivable that many disorders related to exposure to toxic oxygen species might by benefited. Also disorders related to abnormal vascularization, including but not limited to ocular diseases of vascularization.

The invention provides methods of using this OIR model to identify agents which could reduce induced vascular damage, due to for example to oxygen exposure, or to other defects or abnormalities in blood vessels using the assays described herein or other suitable assays. In non-limiting embodiment these agents may inhibit complement activation or recruitment, for example but not limited to agents which target C3, or any of its components, such as the C3a receptor. In certain embodiments the methods comprise exposing an animal, organ, tissue or cell to a first agent, for example but not limited to toxic levels of oxygen, wherein in non-limiting embodiments the levels of oxygen induce damage to blood vessels (disease of vascularization), for example but not limited to capillaries or capillaries beds in the retina, determining the levels of damage to blood vessels in the absence and presence of a second agent. Reduced level of damaged blood vessels in the presence of the second agent is indicative that the second agent can reduce blood vessels damage induced by the first agent. In non-limiting embodiments, any amount of reduction of levels of blood vessels as detected by any suitable method is sufficient to identify the second agent as potentially therapeutic. Methods to determine damage to blood vessels, including quantitative and/or functional assays, are known in the art.

The invention provides methods to treat or prevent ROP by administering to a subject in need thereof an agent which inhibits complement activation or recruitment. The invention provides methods to treat or prevent ROP by administering to a subject in need thereof SB290157.

The invention provides methods to treat other diseases/disorders associated with abnormal blood vessels, including ocular diseases of the retina, by administering complement inhibitors.

The invention provides methods to treat a disease or disorder caused by oxygen toxicity to vascular tissue, for example but not limited to developing blood vessels, comprising administering to a subject in need thereof a therapeutic amount of an agent which inhibits complement activation.

The invention provides methods to treat ocular related conditions due to abnormal ocular/retinal vascularization comprising administering to a subject in need thereof a therapeutic amount of an agent which inhibits complement activation.

The invention provides methods to treat disorders related to exposure to toxic oxygen species, by administering complement inhibitors.

In non-limiting embodiments the complement inhibitors target C3 complement or any of its components, including but not limited to components such as the C3a receptor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simplified schematic of how oxygen applied to the vessel wall cells destroys the polyunsaturated fatty acids which are the major components of the cell wall and breaks the blood retinal barrier thereby activating the C3 Complement protein. The response of the newborn mouse's developing retinal blood vessels to oxygen injury (OIR) is the accepted animal model of human Retinopathy of Prematurity (ROP). ROP is the commonest cause of acquired blindness in human infancy and is now worldwide in distribution. This project investigates the role of the Complement Activation System, specifically Complement C3 protein, as the “first responder” to the injured retinal blood vessels. This has never before been described or reported in the literature describing the effects of oxygen exposure to retinal blood vessels.

FIG. 2 show normal retinal vessel development, FIG. 2A, and loss and damaged retinal vascular development in the oxygen raised pup, FIG. 2B. Note severe loss of vessels in the inner retina in B.

FIG. 3 shows vasculature of normal retina (upper left) and oxygen-exposed retina (upper right), C3 distribution of normal retina (lower left) and oxygen-exposed retina (lower right), demonstrating that C3 is absent in the retina of a mouse raised in normal oxygen while C3 is widely distributed around the blood vessels of retina of an oxygen-exposed mouse.

FIG. 4 shows the structure of SB290157.

FIG. 5 shows DOL 9 (48 hourse in 75% O₂): C3a is present and coats the small vessels after 2 days in 75% O₂.

FIG. 6 shows a DOL 51 mouse retina following 120 hour oxygen exposure. The DOL 51 retina still shows severe damage. C3a is apparent in vessel/lumen.

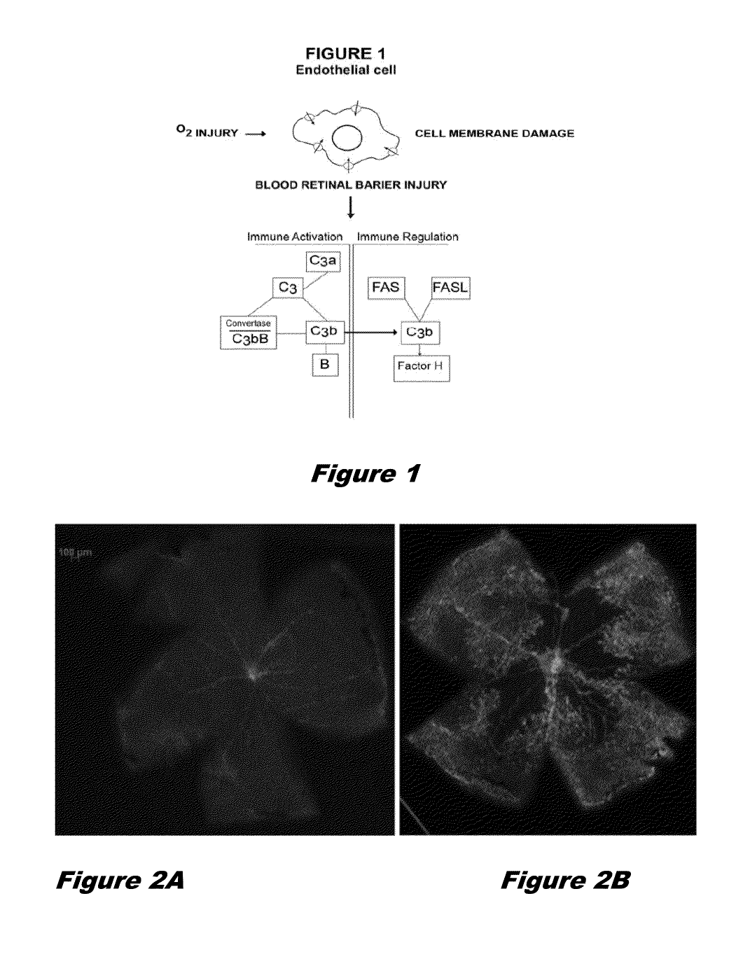

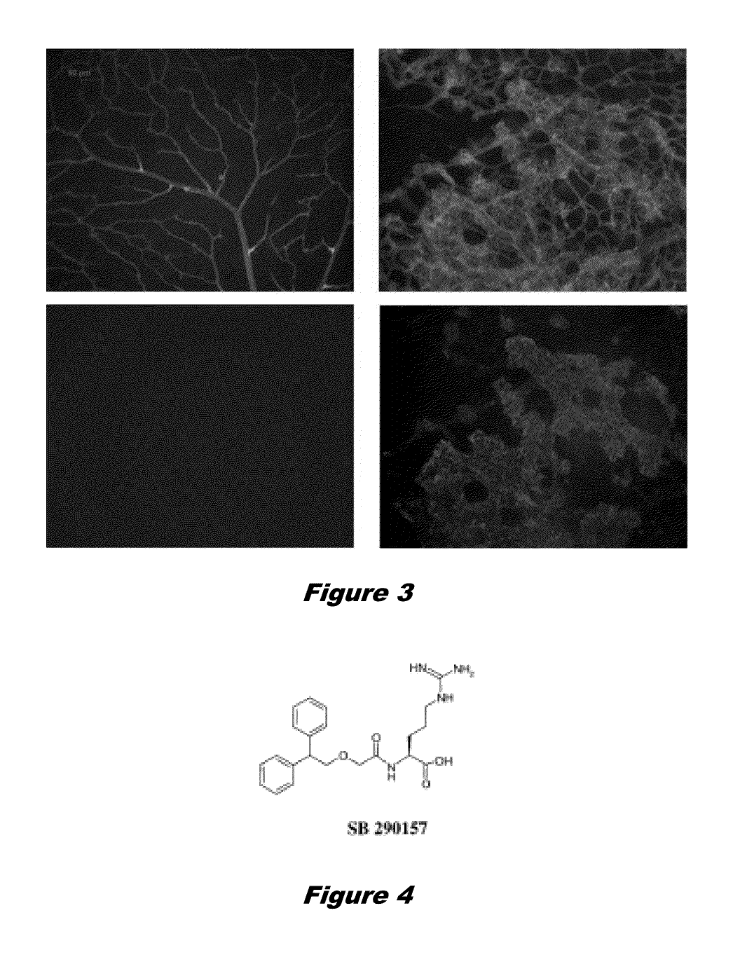

### DETAILED DESCRIPTION

The invention provides evidence of the involvement of complement cascade, including C3a, in the pathogenesis of oxygen-induced retinopathy.

The invention provides methods to modulate the complement pathway by pharmacological agents so as to treat or prevent oxygen-induced retinopathy. In a non-limiting example the pharmacological agent is a C3aR antagonist. A non-limiting example of an agent is SB290157. See Ames et al. (2001) Identification of a selective nonpeptide antagonist of the anaphylatoxin C3a receptor that demonstrates anti-inflammatory activity in animal models. J Immunol 166:6341-8., which is incorporated by reference herewith. Other agents which modulate the complement pathway are synthetic small-molecules inhibitors as disclosed in Holland et al. (2004) “Synthetic small-molecule complement inhibitors” Curr Opin Investig Drugs 5:1164-73, which is incorporated by reference herewith.

Suitable complement inhibitors include antibodies against C1, C2, C3, C4, C5, C6, C7, C8, and C9. In certain embodiments antibodies may include 1) immunoglobulins produced in vivo; 2) those produced in vitro by a hybridoma; 3) antigen binding fragments (e.g., Fab′ preparations) of such immunoglobulins; and 4) recombinantly expressed antigen binding proteins (including chimeric immunoglobulins, bispecific immunoglobulins, heteroconjugate immunoglobulins, “humanized” immunoglobulins, single chain antibodies, antigen binding fragments thereof, and other recombinant proteins containing antigen binding domains derived from immunoglobulins). Such antibodies can include, but are not limited to, polyclonal, monoclonal, humanized, human, bispecific, and heteroconjugate antibodies and can be prepared by applying methods known in the art. See for example; Riechmann et al. (1988) Reshaping human antibodies for therapy. Nature 332:323-7; Winter and Milstein (1991) Man-made antibodies. Nature 349:293-9; Clackson et al. (1991) Making antibody fragments using phage display libraries. Nature 352:624-8; Morrison S L (1992) In vitro antibodies: strategies for production and application. Annu Rev Immunol 10:239-65; Haber E (1992) Engineered antibodies as pharmacological tools. Immunol Rev 130:189-212; Rodrigues et al. (1993) Engineering Fab′ fragments for efficient F(ab)2 formation in Escherichia coli and for improved in vivo stability. J Immunol 151:6954-61.

Methods to humanize antibodies or make human antibodies are known in the art. Methods for humanizing non-human antibodies are well known in the art. Generally, a humanized antibody contains one or more amino acid residues that are introduced from a non-human antibody source. These non-human amino acid residues are often referred to as “import” residues, which are typically taken from an “import” variable domain. Humanization can be essentially performed following the method of Winter and co-workers [Jones et al. (1986) Replacing the complementarity-determining regions in a human antibody with those from a mouse. Nature 321:522-5; Riechmann et al. (1988) Reshaping human antibodies for therapy. Nature 332:323-7; Verhoeyen et al. (1988) Reshaping human antibodies: grafting an antilysozyme activity. Science 239:1534-6], by substituting rodent CDRs or CDR sequences for the corresponding sequences of a human antibody. Humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter (1992) By-passing immunisation. Human antibodies from synthetic repertoires of germline VH gene segments rearranged in vitro. J Mol Biol 227:381-8; Marks et al. (1991) By-passing immunization. Human antibodies from V-gene libraries displayed on phage. J Mol Biol 222:581-97]. The techniques of Cole et al. and Boerner et al. are also available for the preparation of human monoclonal antibodies [Cole et al. (1985) The EBV-hybridoma technique and its application to human lung cancer. In: Reisfeld and Sell (eds) Monoclonal Antibodies and Cancer Therapy. Alan R. Liss, Inc., New York, pp 77-96.; Boerner et al. (1991) Production of antigen-specific human monoclonal antibodies from in vitro-primed human splenocytes. J Immunol 147:86-95].

Similarly, human antibodies can be made by introducing human immunoglobulin loci into transgenic animals, (e.g., mice) in which the endogenous immunoglobulin genes have been partially or completely inactivated. Upon challenge with antigens, only human antibodies are produced in a manner similar to that seen in humans in all respects, including gene rearrangement, assembly, and antibody repertoire. See for example, in U.S. Pat. Nos. 5,545,807; 5,545,806; 5,569,825; 5,625,126; 5,633,425; 5,661,016, and in the following scientific publications; Marks et al. (1992) By-passing immunization: building high affinity human antibodies by chain shuffling. Biotechnology 10:779-83; Lonberg et al. (1994) Antigen-specific human antibodies from mice comprising four distinct genetic modifications. Nature 368:856-9; Morrison S L (1994) Immunology. Success in specification. Nature 368:812-3; Fishwild et al. (1996) High-avidity human IgG kappa monoclonal antibodies from a novel strain of minilocus transgenic mice. Nat Biotechnol 14:845-51; Neuberger M (1996) Generating high-avidity human Mabs in mice. Nat Biotechnol 14:826; Lonberg and Huszar (1995) Human antibodies from transgenic mice. Int Rev Immunol 13:65-93.

Suitable complement inhibitors include RNAi molecules.

The sequences of inhibitory RNAs are based on the sequence of the target gene, and methods to design iRNAs are known in the art. Methods to design and make inhibitory RNAs against a specific target are known in the art. A non-limiting example of such method is described in Schramm & Ramey, “Schramm and Ramey (2005) siRNA design including secondary structure target site prediction. Nature Methods 2, as well as in Chalk and Sonnhammer (2008) siRNA specificity searching incorporating mismatch tolerance data. Bioinformatics 24:1316-7. Many methods have been developed to make siRNA, e.g., chemical synthesis or in vitro transcription. Inhibitory RNA may be synthesized either in vivo or in vitro. Once made, the siRNA can be introduced directly into a cell to mediate RNA interference. The siRNAs can also be introduced into cells via transient or stable transfection, using RNAi expression vectors.

A number of expression vectors have also been developed to continually express siRNAs in transiently and stably transfected mammalian cells. Some of these vectors have been engineered to express small hairpin RNAs (shRNAs), which get processed in vivo into siRNA-like molecules capable of carrying out gene-specific silencing.

In certain embodiments, the iRNAs are “small interfering RNAs” or “siRNAs,” which are known and described in the art. The siRNA may be double stranded, and may include short overhangs at each end. The overhangs are 1-6 nucleotides in length at the 3′ end. It is known in the art that the siRNAs can be chemically synthesized, or derived from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end [Caplen et al. (2001) Specific inhibition of gene expression by small double-stranded RNAs in invertebrate and vertebrate systems. Proc Natl Acad Sci USA 98:9742-7; Elbashir et al. (2001) Functional anatomy of siRNAs for mediating efficient RNAi in Drosophila melanogaster embryo lysate. EMBO J. 20:6877-88]. These double-stranded siRNA structures can then be directly introduced to cells, either by passive uptake or a delivery system of choice, such as described herein. In certain embodiments, the siRNA constructs can be generated by processing of longer double-stranded RNAs, for example, in the presence of the enzyme dicer. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

The sequences of the complement pathway are known in the art and available in public databases.

Suitable complement inhibitors include peptides or peptidomimetics. Non-limiting examples include but are not limited to compstatin and POT-4. Compstatin is a synthetic 13 amino acid cyclic peptide that binds tightly to complement component C3, preventing its participation in the complement activation cascade. As C3 is a central component of all three known complement activation pathways, its inhibition effectively shuts down all downstream complement activation that could otherwise lead to local inflammation, tissue damage and up-regulation of angiogenic factors such as vascular endothelial growth factors. POT-4, a derivative of the cyclic peptide compstatin, is a peptide capable of binding to human complement factor C3 (C3) and prevents its activation, resulting in broad and potent complement activation inhibition. Variants of compstatin are also known in the art. For example, optimized variants are described by Ricklin and Lambris (2008) “Compstatin: a complement inhibitor on its way to clinical application” Adv Exp Med Biol 632:273-92, the content of which publication is herein incorporated by reference in its entirety.

The exact therapeutic amount will be determined by the practitioner, in light of factors related to the subject that requires treatment, and/or a disease or disorder which is treated. Amount and administration are adjusted to provide sufficient levels of the active ingredient or to maintain the desired effect. Factors that may be taken into account include the severity of the disease or disorder, location of the affected tissue or cells within the body, general health of the subject, age, weight, and gender of the subject, diet, time and frequency of administration, drug combination(s), reaction sensitivities, and tolerance/response to therapy. Skilled artisans can readily determine the therapeutic amount which necessary to treat a disease or a disorder, or the therapeutic amount which is necessary to prevent a disease or a disorder.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ formulations suitable for delivery of the complement inhibitory agents of the invention. The methods of the invention employ any suitable route of administration of the therapeutic agents.

Western Blot experiments will be used to determine that C3 is the active complement molecule observed in the oxygen exposed mice in the experiments described.

Experiments will be conducted using C6−/− knockout mice exposed to the same oxygen vs room air wild type mice. The C6−/− knockout mice will be used to assess the involvement of the immune system in the experiments described herein.

### EXAMPLES

### Example 1

### Immunofluorescence Detection of Complement Factor C3 in Murine Oxygen-Induced Retinopathy

Purpose:

To evaluate whether Complement Factor C3 was present in the retinal neovasculature of newborn mice with oxygen-induced retinopathy (OIR). This murine retinopathy is the animal model resembling Retinopathy of Prematurity (ROP) in the human.

Methods:

Litters of newborn mice were divided in half after birth. At seven days of life (DOL) one half of the litter were placed with nursing mothers into an oxygen incubator maintained at 75% oxygen until postnatal day (P)12. The oxygen exposed mice then were removed from the incubator and maintained in room air until P17. The control half of the litter were raised in room air throughout. Both the oxygen exposed mice and room air mice were sacrificed at DOL 17. The eyes were enucleated and fixed in 4% paraformaldehyde at 4° C. overnight. After extraction and washing, the retinas were permeabilized with 0.1% Triton and stained with Alexa Fluor 488-conjugated isolectin GS-IB4, followed by blocking with phosphate buffered saline containing 1% bovine serum albumin (BSA) and 5% donkey serum. The retinas were then incubated sequentially with rat monoclonal antibody against mouse C3 and rhodamine-conjugated donkey anti-rat IgG. Finally, the retinas were flat-mounted on glass slides with cover slips. Images were obtained with a fluorescence microscope and a digital camera.

Results:

By P17, retinal neovascularization was present in all oxygen exposed experimental mice. There were extensive C3 punctate immunostaining throughout the retinas, most prominently in the neovascular tufts and large damaged vessels. No positive C3 staining was found in control subjects. This is the first report of the Complement System involvement in OIR.

Conclusion:

Complement system is involved in the pathogenesis of murine OIR. Additional studies including Western blot quantification, thin section immunostaining and confocal microscopy are underway to elucidate this finding.

### Example 2

### C3a Receptor Antagonist SB290157 Attenuates Murine Oxygen-Induced Retinopathy

As discussed herein, the complement system is involved in the pathogenesis of murine oxygen-induced retinopathy (OIR). Described herein are methods to study the effect of a C3a receptor (C3aR) antagonist, SB290157, a small non peptide molecule, on murine OIR.

Methods:

litters of new born mice were divided into 4 groups. On Day of Life (DOL) 5, SB290157 1 mg per kilogram body weight is injected intraperitoneally into mice in Group 1 and Group 3; same volume solvent without SB290157 is injected intraperitoneally into mice in Group 2 and Group 4. Mice from group 1 and 2 are raised in room air all the time, but mice from group 3 and 4 are raised in room air till DOL 7 when they are transfer into 75% oxygen chamber and on DOL 12 they are returned into room air. On DOL 17, eyes are enucleated and retinas dissected after euthanasia. Retinas are then stained with Alexa Fluor 488 conjugated isolectin GS-IB4 and mounted on slide for vasculature pattern study.

Results:

Retinas from room air raised group 1 and 2 show normal developing vasculature patterns without significant difference. For retinas from oxygen exposed groups, in group 4 which is not SB290157 injected, the retinal whole mounts show extensive capillary wipeout near the optic disc and extensive neovascularization tufts in periphery; in group 3 which is SB290157 injected, there is significantly less capillary damage and less neovascularization tufts all over the whole retinas.

Conclusion:

C3a receptor antagonist, SB290157, can attenuate murine OIR.

### Example 3

### The Role of Complement System in the Pathogenesis of Murine Oxygen-Induced Retinopathy

Retinopathy of Prematurity (ROP) is a serious vasoproliferative disorder affecting premature infants and in its worst cases leading to infant blindness. The disease is worldwide in the developing and developed world with the advent of modern neonatal intensive care in infancy. Low birth weight, prematurity and oxygen exposure are risk factors in its occurrence. The murine OIR is a known animal model for human ROP. This study presents the evidence that active Complement C3a is involved in the pathogenesis of the oxygen injury in newborn mice. This injury results in severe damage and apoptotic loss in extensive areas of the retinal capillary bed. The resulting retinal ischemia leads to neovascularization. Control mice from the same litter raised in room air show no evidence of C3 antibody reactivity, and no such damage and loss of the capillary bed.

Presented herewith is evidence that Complement Factor C3a Receptor Antagonist (C3aRA) SB290157 injected intraperitoneally just prior to the mice being placed in 75% oxygen for 5 days prevents the destruction of capillary bed by the OIR. In certain embodiments, Complement Factor C3a Receptor Antagonist (C3aRA) SB290157 injected intraperitoneally just prior to the mice being placed in 75% oxygen for 5 days reduced the level of destruction of capillary bed by the OIR. The blockade of the active Complement C3a molecule from the endothelial cell membrane receptor in these mice preserves the capillary bed. In the control mice from the same litter who are injected with an equivalent dose of normal saline prior to exposure to 75% oxygen for 124 hours show no such protection from the damage caused by activation of the C3a protein.

Retinopathy of Prematurity (ROP) is a serious vasoproliferative disorder affecting the retinal vascular development of premature infants. In its worst form it causes blindness or severe visual impairment in the infant (1). With the advent of modern neonatal intensive care throughout the developing as well as developed world premature infants in large numbers are surviving. Among them are increasing numbers of micro-prematures (>1000 grams birth weight, 30 weeks gestational age) who are very susceptible to the severe form of ROP and in many cases unresponsive or poorly responsive to laser ablation of the ischemic retinal tissue, the accepted therapy today. This situation calls for an attempt to develop a therapy not aimed at destroying retinal tissue to quench its ischemic signal to save it from the whole panoply of retinal ischemia leading to its eventual death. Instead it would seem the wiser path to seek therapy(ies) preserving the capillary bed which supplies oxygen and metabolites to the retinal neurons and glia removing at the same time the carbon dioxide and waste products. That is the ultimate goal of this work.

Complement, an essential component of the immune system is of substantial importance for the neutralizing and destruction of invading microorganisms and maintaining tissue homeostasis including protection against autoimmune diseases (2, 3). It is in performing this task very much a “first responder” to immune challenge of many types. Following complement C3 activation, biologically active peptides such as C3a and C3b, C5a elicit a number of pro-inflammatory effects including but not limited to recruitment of leukocytes, degranulation of phagocytic, mast and basophilic cells, and increase in vascular permeability. Further amplification results in the generation of toxic oxygen species and the induction and release of arachidonic acid metabolites and cytokines. Under physiological conditions complement activation is effectively controlled by both soluble and membrane bound regulatory proteins. Soluble complement regulators, C1 inhibitor, anaphylotoxin inhibitor, C4b binding protein, Factor H and I among others restrict the action of complement in body fluids at multiple sites of the cascade reaction. Each individual cell is protected against the attack of complement by surface proteins such as Complement receptor 1 membrane cofactor protein (MCP), GPI, DAF, CD59. Improperly activated regulatory mechanisms may be overwhelmed. As a result complement system activation carries considerable risk of harming the host by direct and indirect tissue destruction and disease. Complement has been recognized to play a prominent role in the pathogenesis of numerous inflammatory diseases (4). In addition to immune complex diseases such as rheumatoid arthritis, SLE, ischemia-reperfusion (I/R), SIRS, ARDS, septic shock, burns, trauma, acid aspiration of the lungs, renal diseases, degenerative disease of the nervous system, transplant rejection and inflammatory complication after bypass and hemodialysis. Such a list confirms the role of the Complement System as a “first responder” and mediator in the various disease processes (5-7).

Of recent, more than passing interest to ophthalmology is the reported role of the Complement System may play in Age Related Macular Degeneration, the so called “wet” form of macular degeneration and proliferative form of diabetic retinopathy (8-11). Both entities have shown signs that the immune system and particularly C3 is involved in these adult forms of vascular retinopathies, a previously unsuspected player in their pathogenesis.

Materials & Methods

The mechanism of oxygen injury: The endothelial cell wall of the retinal capillary is composed in large part by lipoprotein molecules composed of side by side arrangement of poly unsaturated fatty acids (12). This structure gives strength and support to the cell wall. In turn this arrangement of unsaturated bonds provides the endothelial cells with the ideal properties to enable it to function as a tightly sealed flexible tube carrying a low velocity, low pressure column of blood to exchange oxygen and nutrients with carbon dioxide and waste product s between the fluid blood and the surrounding neurons. Although the mechanism is not perfectly understood it is known that exposure to 75% oxygen destroys the unsaturated bonds to produce epoxides which are highly reactive and known to lead to the formation of superoxides, singlet oxygen, peroxides and other reactive oxygen species likely to destroy the integrity of the endothelial cell wall (13,14) leading to capillary injury and destruction of the wall's integrity and leakage of intracellular contents outside the cell. The destruction of the cell wall and leakage of cytosolic contents is the signal that precipitates the activation of the C3 cascade.

Murine Model of Oxygen-Induced Retinopathy (OIR): In this regard we reported the role of active C3a in the OIR (15). C57BL/6J mice were obtained from Jackson Laboratories. After birth the litter of mice were nursed by their dam in a room air environment from day of life (DOL) 0 to DOL 6. On DOL 7 the litter were separated in half. One half were placed with their nursing dam in a custom made incubator from DOL 7 through DOL 12 (124 hours) with the oxygen concentration maintained at 75% measured twice daily. The other half of the litter were raised in room air as controls. Dams were replaced every 24 hours in the incubator. On DOL 12 the exposed animals were removed from the incubator and raised in room air with their litter mates. The litter was euthanized at DOL 17 and the eyes enucleated. After the eyes were enucleated 50 μl of 4% paraformaldehyde in phosphate buffered saline was injected into the vitreous. The eyes were then placed in 4% paraformaldehyde in phosphate buffered saline for 12 hours and then switched to 2% paraformaldehyde at 4° C. and stored for future use or dissected immediately. The eyes were opened behind the limbus and the anterior segment, lens, and vitreous removed. Four radial cuts were made through the eye wall and the specimen transferred to a glass slide. The retina was then gently dissected free of the pigment epithelium, choroid and sclera and mounted of the glass slide.

After a rinse, the retinas were permeabilized with 0.1% Triton X100 and stained with Alexa Fluor 488-conjugated isolectin GS-IB4, followed by blocking with 5% donkey serum. The retinas were then subjected to indirect immunofluorescence staining with rat monoclonal antibody against mouse C3 followed by rhodamine-conjugated anti-rat IgG. Finally the retinas were flat mounted on glass slides covered with cover slips. Images were obtained digitally with a fluorescence microscope and a digital camera.

Results:

Low power images of vasculature of whole-mounts in DOL17 retinas show extensive loss of central retinal vascular development in mice subjected to 75% oxygen exposure protocol, compared with mice raised in room air which show no loss of normal retinal vascular development. The area of loss of capillarity is measured by planimetry. Data show that the retina of oxygen exposed mice is devoid of capillaries as compared to control mice raised of room air. Representative images are obtained (6 mice per group).

Medium power images of retinal vasculature of oxygen exposed mice show extensive retinal neovascularization throughout the surviving retina, punctuated by C3 staining of larger damaged vessels and drop out of capillaries in the central retina. Capillaries are presumed lost to apoptosis, in comparison with room air raised mice which show no loss of normal retinal vascular development.

High power images retinal vasculature for room air raised mice show normal retinal vascular development, as compared with oxygen exposed litter mates which show capillary pattern of a neovascular tuft. An image from the same field show C3 staining that is prominent in tufts and large damaged vessels.

Discussion:

The presented evidence supports the role of the active C3a complement molecule at the site of the severe tissue damage and destruction. This is the first report of the Complement System involvement in the OIR. The images show massive injury with total absence of capillaries extending outward from the optic disc in OIR retinas. That is in keeping with the fact that murine capillary development begins in the posterior aspect of the eye sprouting from larger vessels near the optic disc. After DOL 6 (144 hours) the animals are placed in 75% oxygen during DOL 7 through 12 (hours 168 to 292); developing capillaries receive the full brunt of toxic levels of oxygen exposure during this time. They are mortally injured and die. On DOL 12 and thereafter newly formed, but damaged, capillaries, together with the vessel from which they arise, form a very distorted picture with shunts between vessels not clearly identifiable as arteriole or venule and bridged in some areas by malformed capillaries. This picture is reminiscent of the vascular malformations seen in pathology specimens of human ROP. Still further anterior are normal capillary vessels flecked with C3 staining deposits, suggesting the possibility that in these tissues the complement antibody may be playing a protective role. Further study of the retinal circulation with fluorescence dye in vivo and a histological study will be necessary to elucidate this. Additional studies being performed and planned are Western blot for quantification of the C3 presence during a course of OIR development, thin section immunostaining and confocal microscopy for specific tissue localization of C3 deposition, and knock-out mouse modeling of the possibilities of modulating the Complement C3a and other complement molecules involved in the vasculopathy.

Described herein are findings of the role that the complement protein plays in the injury produced by the exposure of newborn capillary endothelium to the 75% oxygen for 120 hours. The damage is profound and the Complement System plays a key early role in removing the damaged and destroyed cells. The C3a protein is attached to the wall of vessels well away from the area of greatest injury and that the duration of its presence in the retina is prolonged after the animal is removed from the oxygen chamber and placed in room air. This is the stage of the OIR where repair of the damage is well along and the neovascular tufts are seen in the in vitreous gel. It supports the idea that complement is a “double edged” sword: not only engaged in reacting to control of damaged tissue but also in repair processes to follow. The OIR model suggests itself as an ideal working model for further study of how the many faceted Complement System may play a significant role in ocular inflammation and repair, heretofore overlooked in eye vasculopathies as well as other ocular diseases.

### REFERENCES FOR EXAMPLE 3

1. Mechoulam H, and Pierce E A (2003) Retinopathy of prematurity: molecular pathology and therapeutic strategies. Am J Pharmacogenomics 3:261-77.
2. Roitt's Essential Immunology. 11th Edition. Blackwell; p11-23, p212-214, p323.
3. Nonaka, M. Phylogeny of the complement system In: Volnakis, J E, Frank, M M, eds. The human complemnt system in health and disease. New York: Marcel Dekker, Inc. 1998, P203-215.
4. Mollnes T E, Song W C, and Lambris J D (2002) Complement in inflammatory tissue damage and disease. Trends Immunol 23:61-4.
5. Walport M J (2001) Complement. First of two parts. N Engl J Med 344:1058-66.
6. Walport M J (2001) Complement. Second of two parts. N Engl J Med 344:1140-4.

7. Szbeni, J (Ed) The Complement System: Novel Roles in Health and Disease. KluwersAcademic Publishers, 2004, MA
8. Hecker L A, and Edwards A O (2010) Genetic control of complement activation in humans and age related macular degeneration. Adv Exp Med Biol 703:49-62.
9. Donoso L A, Vrabec T, and Kuivaniemi H (2010) The role of complement Factor H in age-related macular degeneration: a review. Surv Ophthalmol 55:227-46.
10. Gehrs K M, Jackson J R, Brown E N, Allikmets R, and Hageman G S (2010) Complement, age-related macular degeneration and a vision of the future. Arch Ophthalmol 128:349-58.
11. Yanai R, Thanos A, and Connor K M (2012) Complement involvement in neovascular ocular diseases. Adv Exp Med Biol 946:161-83.
12. Stahl A, Connor K M, Sapieha P, Chen J, Dennison R J, Krah N M, Seaward M R, Willett K L, Aderman C M, Guerin K I, Hua J, Lofqvist C, Hellstrom A, and Smith L E (2010) The mouse retina as an angiogenesis model. Invest Ophthalmol Vis Sci 51:2813-26.
13. Huang H, Van de Veire S, Dalal M, Parlier R, Semba R D, Carmeliet P, and Vinores S A (2011) Reduced retinal neovascularization, vascular permeability, and apoptosis in ischemic retinopathy in the absence of prolyl hydroxylase-1 due to the prevention of hyperoxia-induced vascular obliteration. Invest Ophthalmol Vis Sci 52:7565-73.
14. Hartnett M E (2010) The effects of oxygen stresses on the development of features of severe retinopathy of prematurity: knowledge from the 50/10 OIR model. Doc Ophthalmol 120:25-39.
15. Flynn J T, and Wen Q (2010) Immunofluorescence detection of complement factor C3 in murine oxygen injury retinopathy. Invest Ophthalmol Vis Sci Supp:ARVO:E-Abstract 4469.

### Example 4

### Complement Inhibition Therapy in the OIR

Experiments described herein (1) provide evidence of preservation of capillary bed by C3a Receptor Antagonist SB290157 (2) instead of widespread destruction of the bed by exposure to 75% oxygen from DOL 7-12 (120 hours) in newborn mice. The outcome variable of that exposure showed that while their litter mates control mice raised on room air during that same period showed normal capillary bed development, the oxygen exposed mice showed widespread destruction of the bed and C3a antibody staining to the surviving vessels at the edges of the destroyed bed. There is no C3a antibody staining of any vessels in the room air raised mice. There is marked staining of damaged vessels anterior to the absent capillary bed. The major evidence of damage to the capillary bed is its total absence. The surviving vessels at the edges of capillary absence in the hyperoxia-raised mice show marked vessel abnormalities. Of note is the survival of major vessels crossing the capillary free area. This may support the notion that the major vessel walls are pericyte wrapped and are therefore protected from the oxygen injury specific to the endothelium by excess oxygen. The evidence provided is the first published report of the immune system involvement, and that the complement system plays a direct, not a subsidiary role in the response to the oxygen challenge.

The Choice of Site and Antagonist to block the Complement Cascade

Described herein are experiments to modulate the Complement System by blocking at the level of Complement C3a, the doorway to the complement system (3). This implied a general and broad inhibition of the system leaving till later experiments selective inhibition of active C5a and membrane attack complex C5b-9 (C5b, C6, C7, C8, C9). The strongest argument in favor of the former route chosen was that if Complement activation is the damaging agent it suggests that making the inhibition as broad and complete as possible was the best way to establish this. We were cognizant of the fact that by inhibiting the terminal molecules in the pathway we would be allowing the C3a to assume a defense against the toxic effect of the oxygen injury. We chose the former approach recognizing that as the experimental paradigm unfolded we might in fact find it necessary to reverse course and start site selection deeper in the cascade. There are clearly reasons to pursue either one.

Adverse effects (4): Increased susceptibility to infection and autoimmune disease may be anticipated as effects of blockade of the complement system. Thus far we have observed none of these effects.

C3aR Antagonists: SB290157 (2) (FIG. 4) is a nonpeptide C3a Receptor Antagonist blocking both human and rodent C3aR. It was found to have beneficial effects in airway neutrophilia and to be possibly a beneficial candidate drug in therapy of asthma (2, 5).

Materials & Methods:

Our OIR experiment employing SB290157 differed from the experiments described in Example 3 in that we employed an intraperitoneal injection of 2-5 mg/kg of SB290157 just prior to placing the half litter of mice in 75% oxygen and gave a sham injection of the same size of phosphate buffered saline to the oxygen-exposed half litter.

RESULTS: Calculation of Effect: The effect sought in the SB290157 treated mice is the preservation of capillary bed in the same area where the control sham injected mice sustain acute destruction from oxygen damage. For a given control half litter the areal extent of total destruction can be estimated, in a non-limiting example from the area extending from optic nerve to mid periphery of the retina. For the SB290157 treated animals the measured area of destruction can be estimated, in a non-limiting example from the area extending from optic nerve to mid periphery of the retina. The difference between the two represents the preserved area of capillary bed. The findings indicate that approximately 55% of retinal capillary area is destroyed in a typical newborn exposure to OIR. There are results of three litters exposed to C3aRA (SB290157) as a % of surviving capillaries preserved in the area of expected total destruction. We have not adopted as our measure the standard count of neovascular tufts/high power field (6) because that represents basically a delayed abnormal response to injury far removed from time and space from the injury (capillary destruction-retinal ischemia−VEGF-neovascular tufts). The results of this experiment are measured in prevention of capillary loss. When sufficient capillary bed survives, there is no need for abnormal neovascular development to occur in the absence of an ischemic VEGF signal from retinal neurons starved for oxygen and laden with CO₂. Vascular stain of SB290157 treated animals demonstrates the existing capillary bed in the area which would have been devastated by the exposure to 75% oxygen from DOL 7-12. We employ the presence of the optic nerve in all images as the marker to orient the retinal specimens to the same area of capillary damage.

DISCUSSION: Presented above is the results of a series murine experiments demonstrating the role of a small non-peptide molecule with an affinity for blocking the C3a receptor on the cell membrane's uptake of the C3a molecule. This resulted in preservation of the capillary bed feeding the retinal neurons. Prevention of this loss of capillary bed is the critical first step in blunting the damaging effect of excess ambient oxygen on the capillary bed. By preserving the bed from destruction, the blood supply to the retinal neurons is preserved and the whole chain of events leading to the clinical picture of OIR is aborted.

Fluorescence angiography will be used to investigate the patency/function of that capillary bed to provide oxygen to and remove carbon dioxide to the surrounding neuronal tissues.

### REFERENCES FOR EXAMPLE 4

1. Flynn J T, and Wen Q (2011) C3a receptor antagonism SB 290157 attenuates murine oxygen injury retinopathy. Invest Ophthalmol Vis Sci Supp:ARVO:E-Abstract 3981.
2. Ames R S, Lee D, Foley J J, Jurewicz A J, Tornetta M A, Bautsch W, Settmacher B, Klos A, Erhard K F, Cousins R D, Sulpizio A C, Hieble J P, McCafferty G, Ward K W, Adams J L, Bondinell W E, Underwood D C, Osborn R R, Badger A M, and Sarau H M (2001) Identification of a selective nonpeptide antagonist of the anaphylatoxin C3a receptor that demonstrates antiinflammatory activity in animal models. J Immunol 166:6341-8.
3. Sahu A, and Lambris J D (2001) Structure and biology of complement protein C3, a connecting link between innate and acquired immunity. Immunol Rev 180:35-48.
4. Mollnes T E, and Kirschfink M (2006) Strategies of therapeutic complement inhibition. Mol Immunol 43:107-21.
5. Mizutani N, Nabe T, and Yoshino S (2009) Complement C3a regulates late asthmatic response and airway hyperresponsiveness in mice. J Immunol 183:4039-46.
6. Connor K M, Krah N M, Dennison R J, Aderman C M, Chen J, Guerin K I, Sapieha P, Stahl A, Willett K L, and Smith L E (2009) Quantification of oxygen-induced retinopathy in the mouse: a model of vessel loss, vessel regrowth and pathological angiogenesis. Nat Protoc 4:1565-73.

### Example 5

As described herein, C3a (complement 3a) is involved in the immune response to oxygen injury at DOL (day of life) 17 in the OIR (oxygen-induced retinopathy) mouse model. Described herein is that administration of a C3a receptor antagonist (SB290157) attenuated the damage to the capillary bed in the oxygen-exposed mice in which few to no vascular tufts were noted in the vitreous. No evidence of toxicity to forming vascular tissue was observed in retinas of control mice raised in room air (at normal oxygen concentration).

We now describe series of experiments that will be undertaken to determine the duration from the time of the exposure to 75% oxygen until the time when the C3a molecule is no longer detectable in the retina. This has not been established previously in the OIR mouse model, and yet it is informative to the outcome of work, i.e., its application to the premature infant at risk for ROP (retinopathy of prematurity). Experiments further establish a dosage of SB290157 which will provide the maximum capillary bed survival in the retina without side effects to the animal under hyperoxia conditions.

Provided herein are methods of treatment with a suitable dose of a small molecule receptor blocker (SB290157) which will prevent capillary bed destruction during oxygen exposure in excess of normal ambient concentration (21%) in the infant, preferably employing the maternal vascular system via the placental interface. Methods of determining a suitable dose, for example a therapeutic dose are known to a person of skill in the art. The experiments described herein are designed to find those critical variables, time and concentration, to achieve protection in a mouse OIR model. The mouse OIR model is a model for studying ROP.

Methods:

Experiment 1: Analysis and determining the duration of C3a availability at capillary destruction site. This experiment consists of 2 litters each halved; one half exposed to room air as controls and the other half exposed to 75% oxygen on DOL 7-12. Two pups from controls and oxygen-exposed animals are removed from each arm on DOL 9, 12, 15, 18, 21. Animals are then euthanized, eyes enucleated and retinas prepared for flat mounts. The retinal specimens are then double-stained for vasculature and C3a, the former with fluorescently-labeled isolectin GS-IB4 and the latter with anti C3a antibody and fluorescently labeled secondary antibody.

The images are captured with a fluorescence microscope and digitally recorded in a quantitative manner with known distance and intensity calibration. The extent of the C3a staining is judged in two measures: the area of C3a presence and the immunofluorescence staining intensity. The area of the capillary destruction is determined from the digital images by manually drawing the destruction zone. The C3a intensity is determined by quantitative image acquisition followed by intensity measurements with an image processing program. To provide secure continuity of measurements two more litters are started on the standard room air/oxygen exposure, but the set of measurements commence on DOL 18, 21 (overlapping the first litters' last measurements) 24, 27, 30, 33. The third pair of litters overlap the second, starting on DOL 30, 33, 36, 39, 42, 45, following oxygen exposure on DOL 7-12. The experiments continue until no C3a is observed in the retinas of the oxygen exposed mice. C3a levels (area and intensity) are plotted against DOL, with DOL being the independent variable. An expected outcome of this is that the areal extent of the normal measure surpasses that of the injured retina by approximately 20%, a rough measure of the extent of the damage to the existing retina.

Experiment 2: Analysis to determine a suitable dosage, for example but not limited the lowest dosage (mg of drug per kg of body weight) of C3a receptor antagonist (SB290157) that provides the maximum effectiveness in sparing of the capillary bed. Here we use the experimental paradigm of the OIR mouse model with a quantitative digital image analysis of microscopic retinal images. Two litters of newborn mice are divided into four groups. One half of the litter is raised in room air, and one quarter of that is injected intraperitoneally with 1 mg/kg of SB129157 on DOL 5, and the second quarter is injected with an equivalent volume of diluent. The other half litter is injected intraperitoneally with 1 mg/kg of SB129157 or equivalent volume of diluent as control on DOL 5, then they are exposed to 75% oxygen from DOL 7-12. On DOL 17 the animals are euthanized, the eyes enucleated, the retina removed and flat mounted for processing.

The measurements of retinas of the room air-raised mice are expected to confirm that SB129157 is non-toxic to the retinas at all dosages. We expect that SB129157-treated retinas to show a lesser area of capillary wipe out compared to the sham control, the protective effect being proportional to the dose of SB129157. The experiments are repeated with increasing doses of SB129157, starting at 1 mg/kg until the outcome measures asymptote and no further protection of the capillary bed occurs. At that dosage it is presumed to have reached a plateau of the drug's ability to stave off the complement cascade and perhaps learned the limit of this approach to preventing ROP.

Any other suitable model of ROP can be used to evaluate the therapeutic effect of SB129157, or any other candidate agent. Models of ROP are known in the art, for example as described by Susan E. Yanni and John S. Penn in Chapter 6 “Animal Models of Retinopathy of Prematurity” in “Animal Models for Retinal Diseases”, Editor(s): Iok-Hou Pang, Abbot F. Clark, Series: Neuromethods, Volume: 46, Year: 2010, Page Range: 99-111, the contents of which are hereby incorporated by reference.

### Example 6

### Complement C3a and its Antagonism in Oxygen Injury Retinopathy of Newborn Kittens

Experiments described herein provide evidence of the role of the complement molecule^{1,2}, C3a, played in the acute injury to the developing retina and its blood supply after exposing newborn kittens on day of life (DOL) 7-12 to 75%+/−2% oxygen in a sealed chamber. This injury first described by Smith^{3,4} proved a reliable and reproducible model of the effect of molecular oxygen on neurons, glia and capillary endothelium of the young retina of the mouse. The contribution described herein was the discovery of the very active role played by the molecule, C3a, in the injury and the repair and reconstitution of the retina as the functional visual organ to the animal. The Complement Cascade's⁵⁻⁹ description in other adult retinopathies such as Age Related Macular Degeneration (AMD)¹⁰⁻¹³ and Diabetic Retinopathy(DR)^{14,15} was previously described without a clear pathophysiologic explanation of why the Complement system was involved nor what its specific role might be in the evolution of their lesions. The finding of its presence in the OIR model of ROP suggested a path worth exploring to see what role(s)C3a and its congeners might play in the mouse model known to repair the damage done by the 75% molecular oxygen exposure for 120 hours.

Several questions suggested themselves at this point. What was the location of the active C3a molecule in relation to the developing retinal blood vessels? What was its duration in/on these vessels. Earlier investigations showed that it was at the sites of injury where the edge of vessel malformation and destruction of retinal elements were most active. The C3a presence was most marked at these sites and where capillaries were developing along the edge of the major blood vessels (possibly both arteries and veins). The previous observations described herein related to DOL 17 so the conclusions were drawn with respect to this time axis. At this single time point, it was observed that the large vesseled arteries and veins were spared destruction at this site and did not appear to be invested with C3a at all, whereas surviving capillaries where soaked/coated with the molecule and its counterstained antibody Rhodamine. The first series of experiments therefore, was designed to answer the questions: what, where, when did the complement system appear on the site and for how long did it remain? Answers to these queries are described herein.

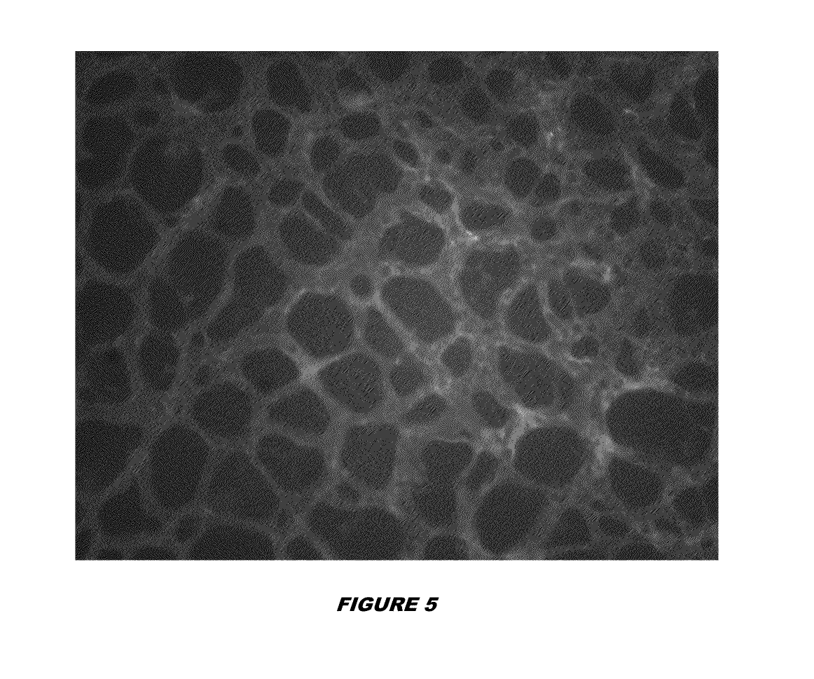

The distribution of the molecule, C3a, over time was first observed at DOL 9 (DOL 9 corresponds to 48 hours in 75% O₂) where it saturated the surviving edges of the capillary beds many of which were deformed (FIG. 5). Larger blood vessels, both arteries and veins were devoid of C3a stain. They seemed cut loose from their moorings, i.e. lying close to, but not in a normal relation to, already damaged but still formed capillary beds. It was expected to find the artery lying with a larger zone clear of capillaries from the nearest bed while the distance from the vein was smaller reflecting the discrepancy in oxygen content in the two systems. However, this is not what was observed. The expected relation of the vessels accompanying the beds was absent. In its place was a tangled mass of vessels at the site, some larger in lumen than capillaries. Some were mere strings of cell wall with no apparent lumens (see below) in proximity as they crossed the areas where both the beds and the expected neurons and glia, as well as capillaries, were gone. It was difficult to distinguish those vessels as arteries or veins. Indeed their lumens seemed flaccid and their course random in contrast to the tight architectural plan followed in normal, room air raised mice controls (room air raised control 9 days). Follow up dates (12, 15, 17, 20 to 51×DOL) confirmed this pattern over time. At first the C3a distributed itself, as was expected, chiefly at the margin of the injured capillary bed. There were two interesting findings noted in these early images of the destruction. In the areas where the destruction was total, i.e. no recognizable vascular or neural elements remaining, there was debris taken to be scattered remnants of cell wall in the area (Maltese Crosses typical of remnants of Poly Unsaturated Fatty Acids (PUFA) known to be integral parts of capillary endothelial cell wall). Without being bound by theory, the unsaturated bonds in the PUFA's produce the flexibility to enable the cell wall to respond to the deformations of flow and velocity of blood with a minimum of turbulence permitting exchange of nutrients and waste products between vessel and tissue). This debris was most notable immediately surrounding the Optic Nerve head and extending outward to the margin of the deformed (possibly dying) capillaries. Viewing this material through crossed polaroid microscopy revealed Maltese Cross images (these are typical of the apoptotic destruction occurring during removal of the Tunica Vasculosa Lentis of the vitreous in human material¹⁵). Without being bound by theory, the exposure to the oxygen rich environment of the chamber oxidizes the unsaturated bonds producing among other species oxides, ethers, aldehydes, hydroxyls and singlet oxygen. Oxidizing the bonds can results in rigidity and destabilizing of the cell wall, preparing it for attack by the Membrane Attack Complex (MAC) consisting of C5-9 elements of the Complement Cascade.

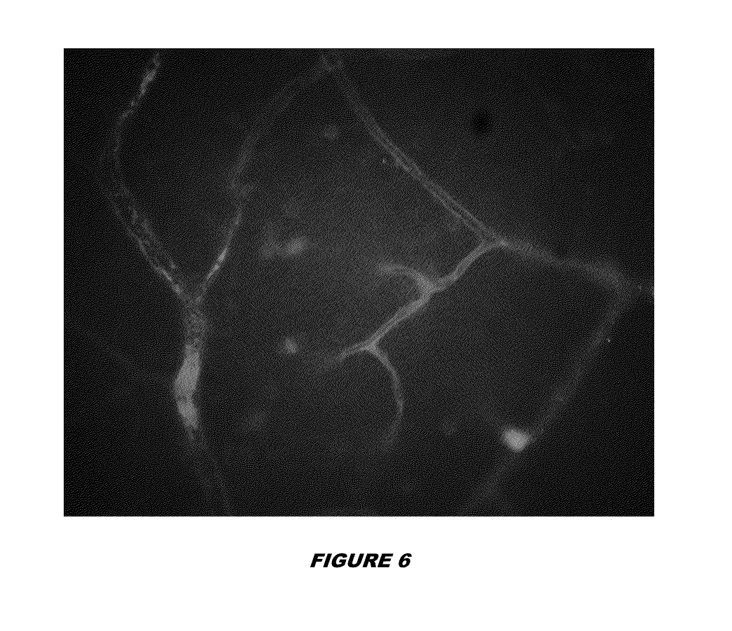

Without being bound by theory, over time after removal of the pups from the oxygen chamber a simultaneous process of demolition and repair of capillaries undergoes at the site of the injury by the Complement Molecule. Of note are two aspects of the OIR. First is collapse of the 3 dimensional structural integrity of the retina unable to sustain its layering. This has been confirmed by confocal microscopy (and the loss can be due to the cell packing by retinal elements and their processes which sustain the spatial separation of the retinal layers. This is most evident when focusing the scope at high power (40×) where little or no re-adjustment of focus in order to bring the deeper layer into focus without clearly losing focus on the superficial layer. The second striking finding is the presence of “ghost capillaries” (as they were called by Cogan in his Doyne' Lecture¹⁶). The most striking change is the loss of luminal diameter. Fluorescian Angiography can be used to show there is no lumen capable of supporting any volume of blood and its elements sufficient to meet the needs of normal retina for nutrients and oxygen. The ghost capillaries can be found in the immediate area of badly damaged vessels. Without being bound by theory, it is believed that the first step in the tissue anoxia leading to excessive VEGF signal and resulting in the abnormal vessel formation leading to the dwindling cycle of invasion of the vitreous gel, proliferation of aberrent membrane supporting the vessels, traction and retinal detachment typical of human ROP. Once this stage, long removed from the inciting injury, is reached the long term prognosis for vision is guarded. Such does not occur in the mouse, but the retinal structure remaining does not truly return to normal (FIG. 6).

This study of the fate of the Complement C3a was based on the time dimension. What happens to the Complement and the bed over time, both during and after the injury (75% Oxygen² X DOL: 5-12) is removed was investigated. Without being bound by theory, it seems that following the terrific wreckage of the early days (up to ˜DOL 20), Complement molecule can acts more in a “clean up the mess” mode, repairing tissue to return to function. It appears that the Complement, and its red stain companion Rhodamine, leaves the site of the major destruction of capillaries and it is found investing neither capillaries nor the Neovacular Tufts which characterize the early recovery from injury. Instead they are found in the larger (but not as large as the major arteries or veins) vessels of intermediate size lumens which are typical of Perforating Vessels bringing or collecting blood from the beds from and to veins and arteries which are the main blood supply to the eye.

To summarize, it has been demonstrated in a series of experiments described herein, the role that Complement C3a plays as ‘first responder” to the defined injury (75% O₂ for 120 hours of exposure from DOLT-12). In addition, it has been shown that the active form of C3a remains chiefly, though not exclusively, at the site of the most severe damage which is total destruction of formed retinal parenchyma extending out from the optic nerve head in a roughly petalloid fashion from the optic disc to the mid periphery of the retina. This massive destruction includes neural and glial elements, as well as all vessels of small (capillary size) caliber, but curiously sparing large arteries and veins, a finding which has been described previously but has not been explained thus far. In that site and its immediate surrounding retina the damage if severe. Distorted vessels, stumps of capillaries and in the total area of destruction many small ill-defined bodies which are not resolved by 40× microscopy. Cross polarized fluorescent microscopy can be used to confirm the theory that the debris is in fact fragments of endothelial wall undergoing apoptosis. The debris will demonstrate Maltese Crosses typical of Polyunsturated Fatty Acids (PUFA) which are a major component of endothelial cell walls. Their linear structure responds to the incident orthogonal light waves in a fashion that results in the exiting waves images of the 90° twist out of phase, producing a cross. The final finding, described herein, is the presence of “ghost vessels” as described by Cogan & Kuabara in Cogan's Doyne lecture which contained the first description of the vascular pathology particular to Diabetic Retinopathy by the newly discovered reti digest by αChymo Trypsin. Cogan described them thus and hypothesized they were remnants of capillaries which displayed this stretched attenuated appearance. This hypothesis will be studied in detail. “Ghost vessels” are found largely clumped around areas of severe capillary bed damage where the rough pentagonal appearance of the beds is also stretched and distorted enough to made it no longer recognizable as a polygonal structure. Without being bound by theory, the assumption of this shape, a regular polygon, is no accident and the capillary bed shape is related to how best to facilitate the transfer of oxygen and nutrients and removal of CO₂ waste. During the study of the preparations that demonstrated the two findings of ghost capillaries and stretched bed architecture, the final finding of this portion of the study was discovered. Namely the collapse of capillary beds in the region of mass destruction. It was discovered that instead of one plane defining a layer of cell bodies at more or less the same depth, the beds piled on top of one another were being imaged. The topmost was in focus while beneath it was another layer at a greater depth focus and therefore a greater depth in the retina. Without being bound by theory, these observations can be explained by the loss of neurons and glial elements packing the spaces tightly and providing the support necessary to produce the layering of the retina into it familiar nine layers.

In parallel with the work on the discovery and investigation of the role of Receptor Antagonists in blocking C3a by way of preventing serious injury to the capillary bed that is just forming during DOL 7-12 weeks gestation, the possibility of blocking the destructive phase of the action of C3a was investigated. In the newborn but otherwise perfectly normal mouse work related to blocking the effect of stroke resulted in an ischemic halo in the mouse model of the human CNS Vascular Accident. Without being bound by theory, the importance that tissue ischemia might play in the CNS, as well as in the eye, was recognized. The C3a Receptor Antagonist (C3aRA), recently discovered by Robert Ames and his group at Glaxo Smith Kline¹⁶, in blocking the effect to antigenic challenge by Anaphalotoxin (another name for C3a) was employed. Without being bound by theory, after the injury is of such magnitude that the body, dealing with this set of circumstances, responds in a vain effort to a keep the challenged cells alive that it is can do nothing but swamp the system with VEGF signal to make more blood vessels. It is from the outset a mismatch between signal ischemia and response non capillary proliferation. The large blood vessels do bring more blood to the site but miss the mark by failing to provide a way for that oxygen bearing blood to transfer it to the cells, neurons and glia desperately needing it. If anything is called for it is a more nuanced signal—one that presumably would provide an environment where the structures, such as the capillaries, could be kept alive through the critical period to provide survival to the neural elements.

A study of this Receptor Antagonist was therefore undertaken, in as gentle and nuanced approach, seeking to preserve capillary architecture and the properties of the individual vessels as much as possible. The damage that excessive oxygen does to the normal capillary bed in a normally birthed newborn mouse was recognized. The experiments were built around the capillary bed, preserving the cells making up its regular polygonal structure first, as well as the cell walls of its endothelium. The architectural integrity rested on these two above mentioned factors Different dosages of SB290157 were employed to investigate if there was any qualitative difference of the specimens after fixation.

The experiments were performed as follows: 1) Route of Injection: Intraperitoneal on DOL 5-6, i.e. the day before placement of the pup in the oxygen chamber; 2) Dosage diminishing while observing the litter carefully for side effects of SB296157 during and after their sojourn in the oxygen: 25 mgm/KG; 17.5 mgm/kg; 12.5 mgm/kg; 5/mgm/kg; 2.5 mgm/kg. Standard O₂ exposure 75% DOL 7-12; 3) Euthanasia at DOL17, followed by enucleation, removal of the retina and separation from the retina from underlying Pigment Epithelial layer. Flatmount and stain for vessels with IsoLectin was performed by incubating the preparation with goat AB followed by counter-staining with Rhodamine to bring out the presence of active C3a; 4)Microscopy at 2.5× and 40× for: capillary bed anatomy; presence of “ghost vessels” previously described; presence of cellular debris noted previously thought to be pieces of partially destroyed but not digested cell wall; destruction or absence of neurons and glial in the retina surrounding the Optic Nerve Head(from which major blood vessels exit undamaged at least as can be detected by the oxygen exposure); presence or absence of neovascular tufts (a late complication of oxygen damage). An additional trial with Intravitreal Injection will be performed as this paper is prepared.

SUMMARY: Described herein is the progress made in efforts to understand the role of C3a in its active form at the site of extensive damage to retinal capillary networks resulting from exposure of newborn mice pups to an atmosphere of 75% O₂ from DOL 7-12. Without being bound by theory, the Complement system appears to be a truly “first responder” to the devastation caused by the oxygen in saturating the unsaturated double bonds of the Poly Unsaturated Fatty Acids (PUFA), the major component of the cell wall, resulting in formation of ethers, oxides, aldehydes and other oxygen species rendering the wall unstable and subject to perforation and loss of cell contents. All these steps leading to cell destruction. On a larger scale it is noted that the regular polygon architecture of the bed was distorted as an integral part of the process and that the damage and destruction by the oxygen resulted in coating of the capillary surface by particulate structures which we identified as antibody stained Complement—Goat AB particles. The capillary bed was further seen to become a distorted shadow of its former self by the presence of “ghost vessels” similar to those identified by David Cogan in Diabetic Retinopathy as part of the picture of the decay of vasculature occurring in the human disease. This similarity supports and strengthens the indication that Complement component of the Immune System is involved in a profound way in the body's immune reaction. This includes reaction to a host of challenges including oxygen toxicity known to play an as yet not understood role in the human disease, Retinopathy of Prematurity. From that starting point in understanding of the role Complement plays in clean up of the damage done by oxygen to the normal newborn mouse's retinal capillaries, the possibility of the development of a Receptor Antagonist SB290157 (C3aRA) was investigated to see if oxygen's destruction of its target organ retinal capillary endothelium could be modulated. The experiments described herein aimed at bringing together Complement's action, modulated by the protection of the C3aRA. This can occur at the cell's wall, thus providing some measure of protection of the cell's PUFA and preventing the wholesale destruction of capillary endothelium and initiating the destruction of the capillary bed and the chain of events leading to the overgrowth of abnormal vessels recognizes as ROP.

MATERIALS & METHOD: Litters of black 6 new born mice were divided in half at birth. At DOL seven, one half of the litter were placed in an oxygen chamber maintained at 75%+/−2% oxygen till DOL 12. The oxygen exposed mice were maintained in room air until DOL 17. The control mice were raised in room air until DOL 17 when both halves were euthanized at DOL17. Eyes were enucleated and fixed in 4% paraformaldehyde at 4° C. overnight. After extraction and washing, the retinas were permeabilized with 0.1% triton-X100 and stained with AlexaFluor 486-conjugated Griffonia simplicifolia Isolectin B4 followed by blocking with PBS containing 1% Bovine Serum Albumin (BSA) and 5% Donkey serum. The retinas were then incubated with rat monoclonal antiboby against mouse C3 and Rhodamine (TRITC) conjugated donkey conjugated anti-rat IgG. Finally the retinas were flat mounted on glass slides with cover slips. Images were obtained with Zeiss Axioplan 2 microscope.

For the trial involving SB290157 litters of new born mice were divided into 2 groups. On DOL5 one half of the litter was injected with SB290157 intraperitoneally while the other half was injected with diluent intraperitoneally. The SB290157dosage was progressively increased from 2.5 mgm/kg to 5, 7.5 10, 15, 25, 50 mgm/kg while the animals were observed for untoward reactions or side effects. None were observed. Both groups were transferred to the O₂ chamber from DOL 7-12, all were removed on DOL 12 and on DOL 17 euthanized and the retinas prepared as before. The slides were scanned for gross differences in neovascular membrane frequency but no count/HPF was performed.

### REFERENCES

1) Flynn, J T, Wen, Q: Immunofluorescence Detection of Complement Factor C3 in Murine Oxygen Injury Retinopathy. ARVO Abstracts.
2) Flynn, J T, Wen, Q:C3a Receptor ANtagonismSB290157AttnuatesMurine Oxygen Retinopathy. ARVO Abstracts.
3) Smith, L E, Wesolowski, E, Mc Lellan, A et al: Oxygen_induced retinopathy in the mouse. Invest Ophthal, Vis Sci:1994, 35(1):101-111.
4) Smith, L
5) Hageman, G et al: A Common Haplotype in the Complement Regulatory Gene Factor H. Predisposes to ARMD. Proc Nat Acad of Science 102.7227, 2005
6) Klein, R J, et al:Complemnt Facrtor HPolumorphisms in ARMDScience, 308,; 385-89, 2005
7) Maller, J Georgein CFHsrtongly enhances risk of Age Related MD. Nature Genetics 65-489, 1963
8) Gerl, V B, Bohl, J, Pitz, S, et al: Extensive deposits of Complement C3d and C5b-9 in the Choriocapillarisof eyes of Patients with Diabetic Retinopathy IOVS 43, (4)2002:1104-08, 2002.
9) Seth, A, Cui, J, To, E: Complemnt—Associated Deposite in the Human Retina, Invest, Ophthalmol, Vis Sci. 2008, 49(2), 743-750, 2008

What is claimed is:

1. A method to treat oxygen-induced retinopathy in a subject comprising administering to a subject in need thereof a therapeutic amount of an agent which inhibits complement activation.

2. A method to prevent oxygen-induced retinopathy in a subject comprising administering to a subject in need thereof a therapeutic amount of an agent which inhibits complement activation.

3. A method to treat oxygen-induced capillary bed damage in the retina of a subject comprising administering to a subject in need thereof a therapeutic amount of an agent which inhibits complement activation.

4. A method to prevent oxygen-induced capillary bed damage in the retina of a subject comprising administering to a subject in need thereof a therapeutic amount of an agent which inhibits complement activation.

5. A method to treat a disease or disorder of abnormal neovascularization due to oxygen induced toxicity in a subject comprising administering to a subject in need thereof a therapeutic amount of an agent which inhibits complement activation.

6. A method to prevent abnormal neovascularization due to oxygen induced toxicity in a subject comprising administering to a subject in need thereof a therapeutic amount of an agent which inhibits complement activation.

7. The method of claim 5 or 6, wherein the abnormal neovascularization is in the eye.

8. A method to treat ROP comprising administering to a subject in need thereof a therapeutic amount of an agent which inhibits complement activation.

9. The method of any of the above claims, wherein the complement targeted for inhibition is C3a receptor.

10. The method of any of any of claims 1-9, wherein the agent is SB290157.

11. A method to treat Retinopathy of Prematurity comprising administering to a subject in need thereof a therapeutic amount of SB290157.

12. The method of claim 11, wherein the subject is a prematurely born infant.

13. A method to identify a therapeutic agent, comprising (a) exposing an animal, organ, tissue or cell to a first agent, wherein the first agent induces damage to blood vessels, and (b) contacting the animal, organ, tissue, or the cell from step (a) with a second agent, and (c) determining the level of damage to blood vessels in the absence and presence of a second agent, wherein reduced level of damaged blood vessels in the presence of the second agent is indicative that the second agent can reduce blood vessels damage induced by the first agent.

14. The method of claim 14, wherein the first agent is oxygen.
